(19) United States
(12) Patent Application Publication
(10) Pub. No.: US 2024/0181129 A1
(43) Pub. Date: Jun. 6, 2024

(54) GUEST-HOST SUPRAMOLECULAR ASSEMBLY OF INJECTABLE HYDROGEL NANOFIBERS FOR CELL ENCAPSULATION

(71) Applicant: University of Virginia Patent Foundation, Charlottesville, VA (US)

(72) Inventors: Steven R. Caliari, Charlottesville, VA (US); Beverly Miller, Charlottesville, VA (US)

(73) Assignee: University of Virginia Patent Foundation, Charlottesville, VA (US)

(21) Appl. No.: 18/277,676

(22) PCT Filed: Feb. 17, 2022
(86) PCT No.: PCT/US2022/016744

§ 371 Date: Aug. 17, 2023

Related U.S. Application Data
(60) Provisional application No. 63/150,328, filed on Feb. 17, 2021.

(51) Int. Cl. A61L 27/20; A61L 27/52; A61L 27/54

(52) U.S. Cl. CPC A61L 27/20; A61L 27/52; A61L 27/54; A61L 2300/252; A61L 2300/62; A61L 2300/64; A61L 2400/06; A61L 2400/12; A61L 2430/10; A61L 2430/30

(57) ABSTRACT

Injectable fibrous hydrogel are provided. The injectable fibrous hydrogels include a guest macromer of a hyaluronic acid (HA) backbone and host macromer of a HA backbone, the guest macromer is a HA electrospun hydrogel nanofiber functionalized with adamantane (Ad), and the host macromer is a HA electrospun hydrogel nanofiber functionalized with β-cyclodextrin (CD). Injectable formulations that include the fibrous hydrogels are also provided, as are methods of making and using the same.

### CROSS-REFERENCE TO RELATED APPLICATIONS

This application is a U.S. National Phase Application of PCT International Patent Application No. PCT/US2022/016744, filed Feb. 17, 2022, incorporated by reference in its entirety, and which claims benefit of U.S. Provisional Patent Application Ser. No. 63/150,328, filed Feb. 17, 2021, the disclosure of which is herein incorporated by reference in its entirety.

### GRANT STATEMENT

This invention was made with government support under Grant Nos. R21AR075181 awarded by the National Institutes of Health and W81XWH-19-1-0157 awarded by the Department of Defense. The government has certain rights in the invention.

### TECHNICAL FIELD

The presently disclosed subject matter is directed to guest-host supramolecular assembly of injectable hydrogel nanofibers for cell encapsulation.

### BACKGROUND

The extracellular matrix (ECM) is a complex three-dimensional (3D) microenvironment that provides mechanical support, protection, and regulatory signals to embedded cells^{1,2}. Hydrogels can mimic native ECM due to their ability to exhibit tissue-like properties, including viscoelastic mechanics and high water content comparable to the tissues they are intended to model and replace³⁻⁵.

While recreating ECM biophysical cues, such as fibrous architecture, has been a focus of the tissue engineering community in recent years³⁰, there remains a need to develop injectable (shear-thinning and self-healing) or in situ gelling materials for both clinical (including drug delivery and tissue engineering with minimally invasive delivery) and additive manufacturing (3D-printing) applications. Clinically, injectable materials offer major advantages such as reduced patient discomfort and treatment cost_{34,35}.

While these needs have been understood for some time, and efforts have been made to address the same, the current solutions fall far short of providing viable compositions suitable for mimicking ECM that are also patient friendly. Thus, there remains an unmet need for fibrous hydrogel systems amenable to minimally invasive delivery. The present disclosure provides such solutions, and demonstrates the superiority of the disclosed injectable fibrous hydrogels based on an analysis of the architecture of the fabricated hydrogel nanofibers, their rheological properties, and their ability to support sustained cell viability following injection.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided herein are injectable fibrous hydrogels, the injectable fibrous hydrogels comprising a guest macromer of a hyaluronic acid (HA) backbone and host macromer of a HA backbone, wherein the guest macromer comprises a HA electrospun hydrogel nanofiber functionalized with adamantane (Ad), wherein the host macromer comprises a HA electrospun hydrogel nanofiber functionalized with β-cyclodextrin (CD). In some embodiments, the HA of the guest macromer and the HA of the host macromer comprises a methacrylated HA (MeHA), wherein the MeHA is Ad-modified to form Ad-MeHA in the guest macromer, wherein the MeHA is CD-modified to form CD-MeHA in the host macromer. In some aspects, the methacrylated HA of the electrospun hydrogel nanofibers is covalently photocrosslinked in the presence of a photoinitiator via ultraviolet (UV) light-mediated radical polymerization. In some embodiments, the guest macromer and host macromer are both hydrophobic and form a stable supramolecular, yet reversible, guest-host interaction.

In some embodiments, the injectable fibrous hydrogel nanofibers are configured to imbibe water upon hydration rather than dissolving. In some aspects, the guest macromer and host macromer have a molar ratio ranging from about 1:1 to about 3:1, optionally about 2:1. In some embodiments, the guest macromers and host macromers of the hydrogel nanofibers associate via hydrophobic supramolecular interactions to form a mechanically robust 3D fibrous hydrogel configured for shear-thinning and self-healing post injection. In some aspects, the guest macromers and host macromers of the injectable fibrous hydrogel have an association constant (Kₐ) of about 1×10⁴ M⁻¹ to about 1×10⁵ M⁻¹, or at least about 1×10⁵ M⁻¹.

In some embodiments, the fibrous hydrogel is flowable through a needle at about 8 mL h⁻¹ to about 20 mL h⁻¹ (using a 16 to 22 gauge needle), optionally at about 12 mL h⁻¹ (16 gauge needle), wherein the fibrous hydrogel is configured to transform to a stable hydrogel plug post injection. The injectable fibrous hydrogel can comprise a self-assembling guest-host fibrous hydrogel configured as a cell carrier for injectable tissue engineering. In some aspects, the injectable fibrous hydrogel can be configured to mimic an extra cellular matrix (ECM) upon injection, optionally wherein the injectable fibrous hydrogel is configured to form a hierarchical assembly upon injection to provide physical cues to cells at different length scales, mimicking the 3D cues provided by a native fibrous ECM. In some embodiments, the injectable fibrous hydrogel further comprises one or more ligands, optionally one or more cell adhesion peptides, optionally a cell adhesion peptide comprising arginylglycylaspartic acid (RGD), and other thiolated molecutles including peptide and fibronectin fragments, to permit integrin-mediated cell adhesion.

Also provided herein in some embodiments are injectable formulations comprising an injectable fibrous hydrogel. Such injectable formulations can further comprise one or more cells encapsulated in the fibrous hydrogel. Any suitable type of cell can be encapsulated, including for example but not limited to, hMSC, myoblasts (C2C12), fibroblasts (3T3), adipose-derived stem cells (ADSC), or pluripotent stem cells (PSC), all with varying applications. The one or more cells can have a post-injection survival rate of at least about 70%, optionally at least about 80%, optionally at least about 90%. In some embodiments, the injectable formulation can be configured for injection into a tissue of a subject, optionally a fibrous tissue, optionally a muscle, tendon, or ligament tissue. In some aspects, the fibrous hydrogel is flowable through a needle at about 8 mL h⁻¹ to about 20 mL h⁻¹ (using a 16 to 22 gauge needle), optionally at about 12 mL h⁻¹ (16 gauge needle), wherein the fibrous hydrogel is configured to transform to a stable hydrogel plug post injection. In some aspects, the injectable formulation comprises one or more pharmaceutically acceptable carriers or excipients. In some embodiments, the injectable formulation comprises a storage modulus (G′) of about 6.6 kPa at 1% fibrous content at 10 Hz, and a G′ of about 9.2 kPa at 5% fibrous content at 10 Hz.

Provided herein in some embodiments are methods of forming a fibrous hydrogel nanofiber in a polymer solution, the method comprising methacrylating a hyaluronic acid (HA) backbone via methacrylate esterification with a primary hydroxyl group of a sodium HA to form methacrylated HA (MeHA); synthesizing adamantane (Ad)-modified MeHA (Ad-MeHA) and β-cyclodextrin (CD)-modified MeHA (CD-MeHA) by anhydrous coupling; electrospinning the fibrous hydrogel nanofiber in the polymer solution; and crosslinking the fibrous hydrogel nanofiber by exposure to ultraviolet (UV) light. In some embodiments, a degree of methacrylate modification of HA is controlled by an amount of a methacrylic anhydride introduced during the methacrylating step, optionally wherein the degree of methacrylate modification of HA is about 10% to about 40%, optionally about 20% to about 30%, optionally about 28%. In some aspects, Ad-MeHA is prepared using 1-adamantane acetic acid via di-tert-butyl bicarbonate (BOC₂O)/4-dimethylaminopyridine (DMAP) esterification, wherein CD-MeHA is prepared using CD-HDA via (benzotriazol-1-yloxy) tris(dimethylamino) phosphonium hexafluorophosphate (BOP) amidation. In some embodiments, the electrospinning comprises a collection plate set-up using an applied voltage of about 9.5-10.5 kV (optionally ranging from about 7.5-12.5 kV), a distance from needle to collector of about 16 cm, a needle gauge of about 20, and a flow rate of about 0.4 mL h⁻¹. In some aspects, crosslinking the fibrous hydrogel nanofiber with UV light comprises exposure to UV light at about 320-390 nm for about 10-15 minutes, optionally about 365 nm for about 15 minutes. In some embodiments, such methods further comprise repeatedly triturating the hydrogel fibers via needle extrusion to produce short fiber segments of a length of about 5 μm to about 20 μm, optionally about 12.7±5.0 μm.

Finally, provided herein in some embodiments are methods of treating a tissue of a subject, the methods comprising providing a subject to be treated and delivering to a tissue of the subject an injectable fibrous hydrogel as disclosed herein. In some embodiments treating the tissue can be a component of treating a wide range of musculoskeletal conditions or diseases, in addition to further tissue applications, e.g. brain, adipose, or skin tissues. In some aspects, the injectable fibrous hydrogel is administered to the tissue to be treated by injection. In some embodiments, the tissue to be treated is selected from a fibrous tissue, optionally a muscle, tendon, or ligament tissue. In some embodiments, the injectable fibrous hydrogel comprises one or more encapsulated cells (varying types of cells are suitable, as discussed further herein). In some aspects, the encapsulated cells have a higher viability post-injection when encapsulated in the fibrous hydrogel than when not encapsulated in the fibrous hydrogel, optionally a survivability rate of at least about 80%.

These and other objects are achieved in whole or in part by the presently disclosed subject matter. Objects of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A-1D are directed to guest-host supramolecular design to make injectable fibrous hydrogels. FIG. 1A depicts structures of guest (Ad-MeHA) and host (CD-MeHA) macromers. The methacrylates enable photocrosslinking to stabilize the fiber structure following electrospinning, while FIG. 1B shows interaction of β-cyclodextrin (CD, host) and adamantane (Ad, guest) groups forms a reversible guest-host inclusion complex to enable shear-thinning and self-healing. FIG. 1C is a SEM of host (CD-MeHA) and guest (Ad-MeHA) electrospun fibers prior to hydration and hydrogel formation. Scale bars=1 μm. FIG. 1D is a schematic of the fibrous hydrogel composed of mixed guest and host fibers decorated with Arginylglycylaspartic acid (RGD) to permit integrin-mediated cell adhesion. The interactions between Ad and CD on complementary fibers result in assembly of a macroscale fibrous hydrogel scaffold.

FIGS. 2A-2B are directed to guest and host fiber morphology and diameter distribution. FIG. 2A shows SEM images of photocrosslinked guest (Ad-MeHA) and host (CD-MeHA) fibers prior to hydration with quantification of dry fiber diameter (n=300 fibers) shown in the right panel. FIG. 2B shows confocal images of hydrated guest (Ad-MeHA) and host (CD-MeHA) fibers and quantification of the swollen hydrogel fiber diameter (n=300 fibers) shown in the right panel.

FIGS. 3A-3D include experimental results showing mechanical integrity as well as shear-thinning and self-healing character of guest-host-assembled fibers. FIG. 3A shows frequency sweep of individual, Ad-MeHA (guest) and CD-MeHA (host), hydrogel fibers and mixed guest-host fibers at constant strain of 0.5%. The storage moduli (G′) of all fiber populations are greater than the loss modulus (G″), reflecting the properties of the photocrosslinked fibers and their ability to entangle. However, the higher moduli of the guest-host hydrogel fiber mixture demonstrates the combined contributions of fiber photocrosslinking and supramolecular interactions between the complementary fiber populations. Additionally, G″ is within an order of magnitude of G′, indicative of the viscoelastic nature of the fibrous hydrogel. FIG. 3B shows qualitative inversion test of mixed guest-host, guest, and host fibers following injection into vials using a 16G needle. When fibers were left inverted for 24 hrs, the mixed guest-host fibers demonstrated long-term mechanical stability and maintenance of their original shape while the guest fibers and host fibers did not. FIG. 3C is a strain sweep of the mixed guest-host fibers showed loss moduli crossover at strains greater than 100%, highlighting the injectability of the fibers. FIG. 3D shows five-step strain sweeps of low strain (0.5%,100 s) and high strain (250%, 100 s). Guest-host fibrous hydrogels show higher loss moduli than storage moduli at high strains and quickly recover viscoelastic properties at low strains, highlighting shear-thinning and self-healing properties.

FIGS. 4A-4D show that injected Human mesenchymal stromal cells (hMSCs) encapsulated in fibrous hydrogels are viable and show increased spreading compared to non-fibrous hydrogels. Human mesenchymal stromal cells were encapsulated in non-fibrous MeHA, non-fibrous guest-host, and fibrous guest-host hydrogels. Live/Dead images of viable and membrane damaged cells as well as cell viability quantification for all groups is shown (FIG. 4A) immediately following injection (Day 0), (FIG. 4B) after 3 days of culture, and (FIG. 4C) after 7 days of culture. Inset image scale bar=10 μm. Full image scale bar=100 μm. Cell viability was comparable across the different hydrogel formulations. FIG. 4D shows quantification of day 7 cell shape metrics. Cells in fibrous guest-host hydrogels showed significantly increased projected cell area and elongation compared to cells in MeHA hydrogels. Additionally, hMSCs in fibrous guest-host hydrogels showed significantly reduced cell shape index (circularity) compared to hMSCs in non-fibrous guest-host hydrogels. Viability data are presented as mean+/−SD. Tukey box plots of individual cell data (60 cells per group) show the second and third quartiles as boxes, the median as a line between the boxes, and error bars with the lower value of either 1.5 times the interquartile range or the maximum/minimum value. Data points outside this range are shown individually. * P<0.05, ** P<0.01, *** P<0.001, **** P<0.0001. n=at least 6 hydrogels per experimental group.

FIG. 5 is a ¹H NMR spectrum of methacrylate-modified hyaluronic acid (MeHA). The degree of HA modification with methacrylates was determined to be 28%. Degree of functionalization was determined from integration of the methylene (δ=5.82, 1H and δ=6.25, 1H) labeled ‘1’ relative to the HA backbone, (δ=3.10-4.10, 10H).

FIG. 6 is a ¹H NMR spectrum of methacrylated hyaluronic acid tert-butyl ammonium salt (MeHA-TBA). The MeHA used for the synthesis of Ad (guest) and CD (host) derivatives underwent addition of the TBA salt (labeled ‘2’) to enable further HA modification.

FIG. 7 is a 1H NMR spectrum of adamantane and methacrylate-modified hyaluronic acid (Ad-MeHA). The degree of MeHA modification with adamantane was determined to be 43%. The integration of the ethyl multiplet of adamantane (δ=1.40-1.70, 12H) labeled ‘2’ gives the degree of modification relative to the HA backbone (δ=3.10-4.10, 10H).

FIG. 8 is a 1H NMR spectrum of β-cyclodextrin and methacrylate-modified hyaluronic acid (CD-MeHA). The degree of MeHA modification with β-cyclodextrin was determined to be 26%. This was calculated from the integration of the hexane linker (δ=1.20-1.75, 12H) labeled ‘2’ relative to the methacrylate modification, which is assumed to stay constant.

FIG. 9 shows fiber length distribution following trituration. The lengths of rhodamine-labeled Ad-MeHA ‘guest’ and CD-MeHA ‘host’ fibers were separately evaluated following sequential needle trituration. Fiber lengths (n=90) were measured using confocal images and processed in ImageJ.

FIG. 10 shows the rheological properties of the guest-host fiber network with varying fiber density. The frequency-dependent behavior was measured using a constant strain of 0.5%. The 1% fibrous hydrogel reached a final storage modulus (G′) of 6.6 kPa and the 5% fibrous hydrogel a G′ of 9.2 kPa at 10 Hz.

FIG. 11 shows the rheological properties of the non-fibrous MeHA and guest-host hydrogels used for cell encapsulation. The frequency-dependent behavior was measured using a constant strain of 0.5%. The non-fibrous 3% MeHA hydrogel formulation reached a final storage modulus (G′) of 4.2 kPa and the non-fibrous 3% guest-host hydrogel formulation reached a final G′ of 3.7 kPa at 10 Hz.

FIG. 12 is a visualization of the fibrous guest-host hydrogel structure. Rhodamine-labeled HA fibers show the morphology of the fibrous guest-host hydrogel without encapsulated cells (left panel) and hMSCs encapsulated within the fibrous guest-host hydrogel (center panel) after 7 days of culture (enlarged insets (50 um) shown on far right).

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

A “test” cell, tissue, sample, or subject is one being examined or treated.

A “compound”, as used herein, refers to any type of substance or agent that is commonly considered a drug, or a candidate for use as a drug, combinations, and mixtures of the above, as well as other non-limiting examples like polypeptides and antibodies.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “ingredient” refers to any compound, whether of chemical or biological origin, that can be used the disclosed compositions and formulations. The terms “component”, “nutrient”, “supplement”, and ingredient” can be used interchangeably and are all meant to refer to such compounds.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

“Plurality” means at least two.

The term “subject,” as used herein, generally refers to a mammal. Typically, the subject is a human. However, the term embraces other species, e.g., pigs, mice, rats, dogs, cats, or other primates. In certain embodiments, the subject is an experimental subject such as a mouse or rat. The subject may be a male or female. The subject may be an infant, a toddler, a child, a young adult, an adult or a geriatric.

A subject under the care of a physician or other health care provider may be referred to as a “patient”.

A “subject” of diagnosis or treatment is an animal, including a human. It also includes pets and livestock.

As used herein, a “subject in need thereof” is a patient, animal, mammal, or human, who will benefit from the compositions, formulations and methods of the presently disclosed subject matter.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

### II. Injectable Fibrous Hydrogels

The extracellular matrix (ECM) is a complex three-dimensional (3D) microenvironment that provides mechanical support, protection, and regulatory signals to embedded cells^{1,2}. Hydrogels can mimic native ECM due to their ability to exhibit tissue-like properties, including viscoelastic mechanics and high water content comparable to the tissues they are intended to model and replace^{3,5}. In the context of tissue engineering, there is growing appreciation for the importance of the fibrillar architecture of ECM and its role in providing biophysical cues that regulate cell behavior⁶⁻⁹. In particular, there is a need for robust 3D hydrogels that can mimic the fibrous networks present in many musculoskeletal tissues while allowing tuning of network biophysical and biochemical properties. A method of forming mechanically robust fibrous materials is electrospinning, a simple, scalable, and cost-effective biofabrication technique that produces nanofibers with tunable physicochemical properties. Electrospinning has also garnered interest owing to its ability to produce nanofibers.

While recreating ECM biophysical cues, such as fibrous architecture, has been a focus of the tissue engineering community in recent years³⁰, there remains a need to develop injectable (shear-thinning and self-healing) or in situ gelling materials for both clinical (including drug delivery and tissue engineering with minimally invasive delivery) and additive manufacturing (3D-printing) applications. Clinically, injectable materials offer major advantages such as reduced patient discomfort and treatment cost^{34,35}.

Guest-host (e.g., adamantane-β-cyclodextrin) supramolecular chemistries are considered herein for injectable hydrogel design due to their ability to support shear-thinning and self-healing behavior while also allowing careful tuning of viscoelastic mechanical properties through both primary and secondary crosslinking mechanisms. Guest-host assembled hydrogels can also be suitable cell carriers for injectable delivery since their non-Newtonian properties help protect cells from excessive shear during needle extrusion. However, achieving fibrillar topographies in mechanically robust injectable hydrogels remains challenging. While self-assembling peptides can serve as injectable nanofibrous hydrogels⁴⁵, these materials are often limited by poor long-term mechanical properties^{5,8}. Electrospun hydrogel materials, as described earlier, show robust mechanics and support biomimetic cellular behaviors. However, electrospun scaffolds typically require physical implantation and are not amenable to injectable delivery^{46,47}.

To address the need for a fibrous hydrogel system amenable to minimally invasive delivery, disclosed herein are studies and results based on a unique combination of strategies in biomanufacturing, supramolecular chemistry, and tissue engineering to produce a mechanically robust injectable fibrous hydrogel capable of supporting cell encapsulation. The engineering design brings together hyaluronic acid (HA) electrospun hydrogel nanofibers, functionalized with either guest (adamantane, Ad) or host (β-cyclodextrin, CD) supramolecular moieties, that when joined together create a shear-thinning and self-healing hydrogel fiber network via guest-host complexation. HA was utilized for its amenability to functionalization with reactive groups enabling both photocrosslinking and guest-host assembly. In this work the architecture of the fabricated hydrogel nanofibers, their rheological properties, and their ability to support sustained cell viability following injection were assessed.

More particularly, as disclosed and discussed in the working Examples herein, the disclosed injectable fibrous hydrogels can comprise a guest macromer of a hyaluronic acid (HA) backbone and host macromer of a HA backbone, wherein the guest macromer comprises a HA electrospun hydrogel nanofiber functionalized with adamantane (Ad), wherein the host macromer comprises a HA electrospun hydrogel nanofiber functionalized with β-cyclodextrin (CD). The HA of the guest macromer and the HA of the host macromer can include a methacrylated HA (MeHA), wherein the MeHA is Ad-modified to form Ad-MeHA in the guest macromer, and the MeHA is CD-modified to form CD-MeHA in the host macromer. In some aspects, the methacrylated HA of the electrospun hydrogel nanofibers is covalently photocrosslinked in the presence of a photoinitiator via ultraviolet (UV) light-mediated radical polymerization. Notably, the guest macromer and host macromer are both hydrophobic and form a stable supramolecular, yet reversible, guest-host interaction.

The injectable fibrous hydrogel nanofibers can be configured to imbibe water upon hydration rather than dissolving. The guest macromer and host macromer can be optimized as needed and can in some aspects have a molar ratio ranging from about 1:1 to about 3:1, optionally about 2:1. In some embodiments, the guest macromers and host macromers of the hydrogel nanofibers associate via hydrophobic supramolecular interactions to form a mechanically robust 3D fibrous hydrogel. Advantageously, particularly as compared to existing hydrogels, the disclosed compositions are shear-thinning and self-healing post injection. In some aspects, the guest macromers and host macromers of the injectable fibrous hydrogel have an association constant (Kₐ) of about 1×10⁴ M⁻¹ to about 1×10⁵ M⁻¹, or at least about 1×10⁵ M⁻¹.

Importantly, the fibrous hydrogels disclosed herein are flowable through a needle at about 12 mL h⁻¹ (via a 16 gauge needle). The disclosed fibrous hydrogels can transform to a stable hydrogel plug post injection. The injectable fibrous hydrogel can comprise a self-assembling guest-host fibrous hydrogel configured as a cell carrier for injectable tissue engineering. In some aspects, the injectable fibrous hydrogel can be configured to mimic an extra cellular matrix (ECM) upon injection, optionally wherein the injectable fibrous hydrogel is configured to form a hierarchical assembly upon injection to provide physical cues to cells at different length scales, mimicking the 3D cues provided by a native fibrous ECM. The injectable fibrous hydrogels can further include one or more ligands, optionally one or more cell adhesion peptides, optionally a cell adhesion peptide comprising arginylglycylaspartic acid (RGD), and other thiolated molecutles including peptide and fibronectin fragments, to permit integrin-mediated cell adhesion.

The disclosed hydrogels can also be incorporated into injectable formulations. Such injectable formulations can further comprise one or more cells encapsulated in the fibrous hydrogel. Any suitable type of cell can be encapsulated, including for example but not limited to, hMSC, myoblasts (C2C12), fibroblasts (3T3), adipose-derived stem cells (ADSC), or pluripotent stem cells (PSC), all with varying applications. The one or more cells can have a post-injection survival rate of at least about 70%, optionally at least about 80%, optionally at least about 90%. In some embodiments, the injectable formulation can be configured for injection into a tissue of a subject, optionally a fibrous tissue, optionally a muscle, tendon, or ligament tissue. In some aspects, the fibrous hydrogel is flowable through a needle at about 12 mL h⁻¹ (16 gauge needle), wherein the fibrous hydrogel is configured to transform to a stable hydrogel plug post injection. In some aspects, the injectable formulation comprises one or more pharmaceutically acceptable carriers or excipients. In some embodiments, the injectable formulation comprises a storage modulus (G′) of about 6.6 kPa at 1% fibrous content at 10 Hz, and a G′ of about 9.2 kPa at 5% fibrous content at 10 Hz.

Provided herein, and as detailed further in the working Examples, are methods of forming a fibrous hydrogel nanofiber in a polymer solution, the method comprising methacrylating a hyaluronic acid (HA) backbone via methacrylate esterification with a primary hydroxyl group of a sodium HA to form methacrylated HA (MeHA); synthesizing adamantane (Ad)-modified MeHA (Ad-MeHA) and β-cyclodextrin (CD)-modified MeHA (CD-MeHA) by anhydrous coupling; electrospinning the fibrous hydrogel nanofiber in the polymer solution; and crosslinking the fibrous hydrogel nanofiber by exposure to ultraviolet (UV) light. In some embodiments, a degree of methacrylate modification of HA is controlled by an amount of a methacrylic anhydride introduced during the methacrylating step, optionally wherein the degree of methacrylate modification of HA is about 10% to about 40%, optionally about 20% to about 30%, optionally about 28%. In some aspects, Ad-MeHA is prepared using 1-adamantane acetic acid via di-tert-butyl bicarbonate (BOC₂O)/4-dimethylaminopyridine (DMAP) esterification, wherein CD-MeHA is prepared using CD-HDA via (benzotriazol-1-yloxy) tris(dimethylamino) phosphonium hexafluorophosphate (BOP) amidation. In some embodiments, the electrospinning comprises a collection plate set-up using an applied voltage of about 9.5-10.5 kV, a distance from needle to collector of about 16 cm, a needle gauge of about 20, and a flow rate of about 0.4 mL h⁻¹. In some aspects, crosslinking the fibrous hydrogel nanofiber with UV light comprises exposure to UV light at about 320-390 nm for about 10-15 minutes, optionally about 365 nm for about 15 minutes. In some embodiments, such methods further comprise repeatedly triturating the hydrogel fibers via needle extrusion to produce short fiber segments of a length of about 5 μm to about 20 μm, optionally about 12.7±5.0 μm.

Finally, provided herein are methods of treating a tissue of a subject, the methods comprising providing a subject to be treated and delivering to a tissue of the subject an injectable fibrous hydrogel as disclosed herein. In some embodiments treating the tissue can be a component of treating a wide range of musculoskeletal conditions or diseases, in addition to further tissue applications, e.g. brain, adipose, or skin tissues. In some aspects, the injectable fibrous hydrogel is administered to the tissue to be treated by injection. In some embodiments, the tissue to be treated is selected from a fibrous tissue, optionally a muscle, tendon, or ligament tissue. In some embodiments, the injectable fibrous hydrogel comprises one or more encapsulated cells. In some aspects, the encapsulated cells have a higher viability post-injection when encapsulated in the fibrous hydrogel than when not encapsulated in the fibrous hydrogel, optionally a survivability rate of at least about 80%.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-3

Materials. Sodium hyaluronate (sodium HA, 64 kDa) was purchased from Lifecore Biosciences. β-cyclodextrin (CD), hexamethylenediamine (HDA), ammonium chloride, and β-Toluenesulfonyl chloride were purchased from TCI America. Tetrabutylammonium hydroxide (TBA-OH) was purchased from Acros Organics. All other materials were purchased from Sigma-Aldrich.

Synthesis of β-CD-HDA. Synthesis of 6-(6-aminohexyl)amino-6-deoxy-β-cyclodextrin (β-CD-HDA) was performed as previously described^{34,42}, first via synthesis of the intermediate 6-o-monotosyl-6-deoxy-β-cyclodextrin (CD-Tos) using β-cyclodextrin (β-CD) and p-toluenesulfonyl chloride (TosCl). Briefly, β-CD was suspended in water and cooled to 0° C. TosCl was dissolved in minimal acetonitrile and added dropwise. After stirring at room temperature for 2 h, sodium hydroxide was added dropwise. The reaction was stirred at room temperature for 30 min before the addition of solid ammonium chloride to obtain a pH of 8.5. The solution was cooled on ice and the precipitate collected. The CD-Tos product was washed first with cold deionized (DI) water and then cold acetone before being dried under vacuum. After product confirmation using ¹H NMR (500 MHz Varian Inova 500), a round bottom flask was charged with the CD-Tos, HDA, and dimethyl formamide (DMF). The reaction vessel was purged with nitrogen and then the reaction was allowed to proceed with constant nitrogen flow at 80° C. for 18 h. The product was precipitated in cold acetone (5×50 mL/g CD-Tos), washed with cold diethyl ether (3×100 mL), and dried. The degree of modification was determined by 1H NMR.

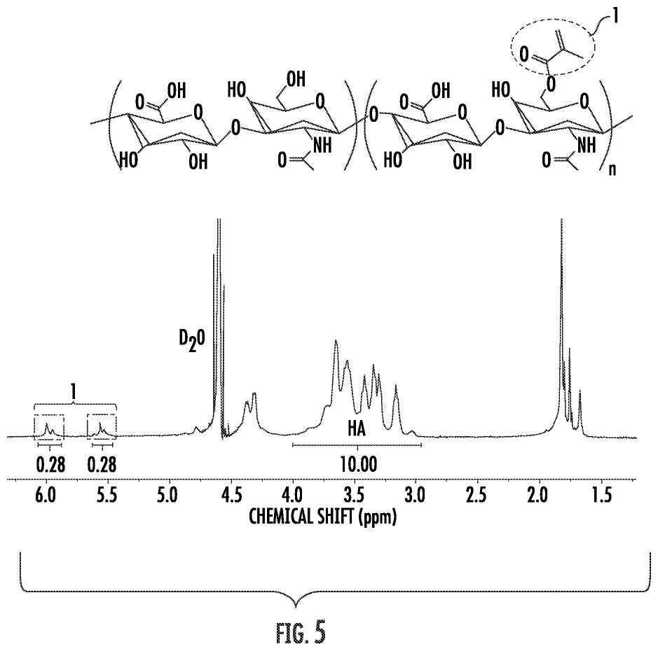

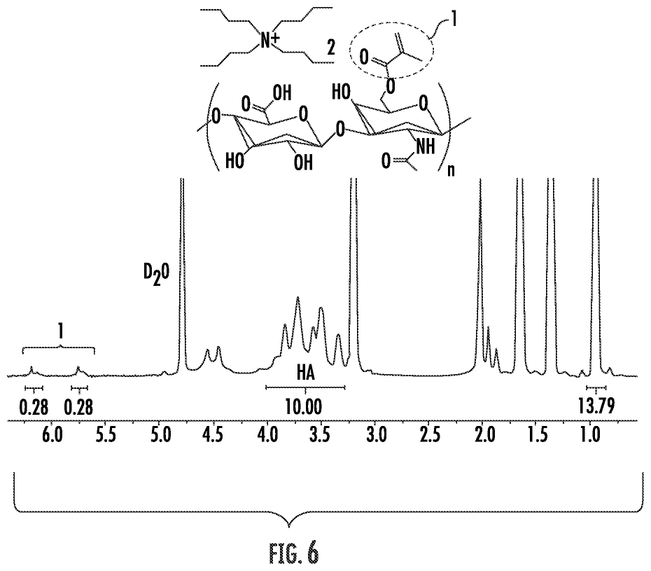

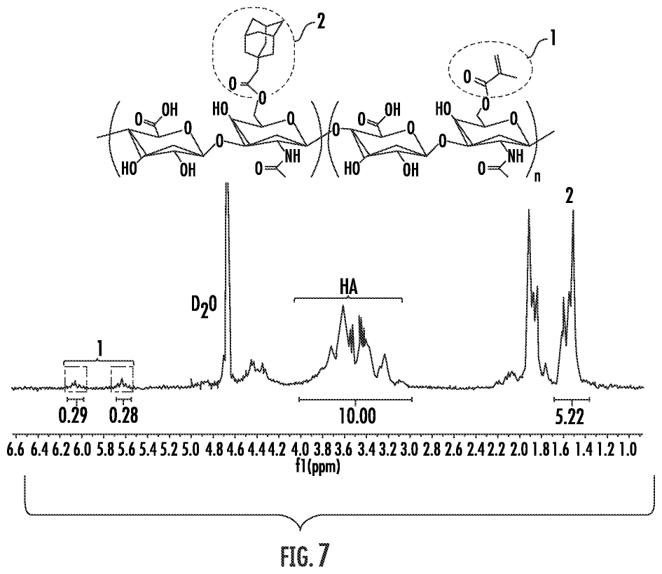

Synthesis of Ad-MeHA and CD-MeHA. Methacrylate modification of the HA backbone was achieved via methacrylate esterification with the primary hydroxyl group of sodium HA at basic pH^{48,49}. The degree of methacrylate modification was controlled by the amount of methacrylic anhydride introduced during synthesis and was determined to be 28% of the HA disaccharides via ¹H NMR (FIG. 5). Next, the methacrylated HA (MeHA) was reacted with proton exchange resin and titrated with TBA-OH to yield methacrylated hyaluronic acid tert-butyl ammonium salt (MeHA-TBA) as previously described 34 (FIG. 6). Next, Ad-modified MeHA (Ad-MeHA) and β-CD-modified MeHA (CD-MeHA) were synthesized⁴⁹ by anhydrous coupling. Ad-MeHA was prepared using 1-adamantane acetic acid via di-tert-butyl bicarbonate (BOC₂O)/4-dimethylaminopyridine (DMAP) esterification while CD-MeHA was prepared using CD-HDA via (benzotriazol-1-yloxy) tris(dimethylamino) phosphonium hexafluorophosphate (BOP) amidation. Products were dialyzed against DI water and degree of modification was determined using ¹H NMR. Esterification of MeHA with 1-adamantane acetic acid resulted in 43% adamantane modification (by ¹H NMR, FIG. 7) while coupling β-CD-HDA to MeHA via amidation resulted in 26% of the HA disaccharides modified with β-cyclodextrin (by ¹H NMR, FIG. 8).

Electrospinning. Ad-MeHA, or CD-MeHA, was dissolved at 2% (w/v) in DI water along with 3.5% (w/v) poly(ethylene oxide) (PEO, 900 kDa) and 0.05% (w/v) Irgacure 2959 (I2959) for 24-48 h prior to electrospinning. The polymer solutions were electrospun using a Spraybase (Kildare, Ireland) collection plate set-up and using the following collection parameters: applied voltage: 9.5-10.5 kV, distance from needle to collector: 16 cm, needle gauge: 20, flow rate: 0.4 mL h⁻¹. Hydrogel nanofibers were deposited onto foil covering the collector plate, placed into a container which was purged with nitrogen, and crosslinked with UV light (365 nm) for 15 minutes. Electrospinning parameters were chosen based on previous work with MeHA⁵⁰ and CD-MeHA⁵¹.

Nanofiber imaging and morphological characterization. To measure the diameters of the Ad-MeHA and CD-MeHA fibers, samples were electrospun onto foil. After electrospinning, samples were photocrosslinked and analyzed in both dry and swollen states. Dry fibers were imaged using scanning electron microscopy (SEM, FEI Quanta 650) at a magnification of 10,000×. To visualize swollen fibers, a methacrylated rhodamine dye (MeRho, Polysciences, 2 mM) was incorporated prior to electrospinning. Rhodamine-labeled fibers were hydrated, and broken up via trituration through increasingly smaller needle gauges (16G-30G) before encapsulation in a 2% (w/v) MeHA hydrogel to facilitate image analysis. Fibers were encapsulated at 0.2% (w/v) for hydrated fiber diameter quantification and at 0.05% (w/v) for post-trituation fiber length quantification. The hydrated fibers were allowed to equilibrate within the MeHA hydrogels overnight in PBS before being imaged using confocal microscopy (Leica inverted confocal microscope, DMi8). Fiber diameters (n=300 fibers per group) and fiber lengths (n=90) were quantified from the resulting SEM (dry fibers) and confocal images (hydrated fibers) using ImageJ (NIH). The corresponding violin plots were made using R. The fiber mass swelling ratio Q_{M} for the Ad-MeHA and the CD-MeHA hydrogel fibers was calculated using the equation:

$${Q}_{M} = \frac{{M}_{w} - {M}_{d}}{{M}_{d}} \qquad Equation 1$$

where M_{w} is the fiber wet mass and M_{d} is the fiber dry mass. Swelling ratios were measured in triplicate for each fiber type.

Hydrogel formulations. Collected guest Ad-MeHA and host CD-MeHA fibers were hydrated (0.1% w/v in DI water) overnight at 37° C. to remove PEO from the electrospinning process⁵², centrifuged, supernatant discarded, and then lyophilized. Once dry, the fibers were again hydrated at 0.1% w/v in DI water, allowed to swell at 37° C. for at least 2 hours, and then passed through needles of progressively smaller gauge sizes via trituration. Starting with a 16G needle and progressing up to a 30G needle, the fibers were passed through the needles until the fiber solution could easily pass through the needle. This process separated any adjoined fibers and resulted in a reproducible fiber suspension. Fiber density was calculated as the weight percentage of the dry fiber mass per volume solvent (w/v). When determining the volume, the dry fibers were first hydrated in a known volume of DI water and allowed to swell overnight. Hydrated fibers were then centrifuged to remove excess liquid. Finally, water was added back to the hydrated fibers to achieve the desired w/v density²⁸. For mechanical testing, the complementary guest and host hydrogel fibers were gently mixed together directly on the rheology plate to create the mixed fibrous guest-host network. Non-fibrous MeHA and guest-host (Ad-MeHA/CD-MeHA) hydrogel groups were prepared as 3% (w/v) solutions and underwent covalent crosslinking (for mechanical stabilization) via photopolymerization in the presence of UV light (365 nm, 10 mW cm⁻²) and 1 mM lithium acylphosphinate (LAP) photoinitiator for 5 min. LAP was used as the photoinitiator for cell culture studies due to its ability to facilitate increased polymerization rates under cytocompatible longer wavelength (365-405 nm) light, even at lower intensities, when compared to 12959⁵³. However, since dense electrospinning solutions take 1-2 days to homogenize (and since cells are not present in electrospinning), it is advantageous to use the less efficient photoinitiator 12959 to prevent solution gelation prior to electrospinning.

Rheology. All rheological measurements were performed on an Anton Paar MCR 302 rheometer with the plate temperature set at 37° C. The non-fibrous MeHA and guest-host hydrogels were characterized using a cone-plate geometry (25 mm diameter, 0.5°, 25 μm gap) immediately following photopolymerization while guest-host fibrous hydrogels were characterized using a parallel-plate geometry (25 mm diameter, 25 μm gap). Mechanical properties for the non-fibrous MeHA and guest-host hydrogel groups were tested using oscillatory frequency sweeps (0.1-10 Hz, 0.5% strain). Fibrous guest-host hydrogels mechanical properties were tested using oscillatory frequency sweeps (also 0.1-10 Hz, 0.5% strain), strain sweeps, and cyclic deformation tests alternating between 0.5% and 250% strain to assess shear-thinning and self-healing capabilities⁴².

Cell culture. Human mesenchymal stromal cells (hMSCs, Lonza) were used at passage 7 for all experiments. Culture media contained Gibco minimum essential medium (MEM-α) supplemented with 20 v/v % fetal bovine serum (Gibco) and 1 v/v % penicillin/streptomycin/amphotericin B (1000 U/mL, 1000 μg/mL, and 0.25 μg/mL final concentrations, respectively, Gibco). Prior to cell encapsulation, all materials were modified with thiolated Arginylglycylaspartic acid (RGD) peptide (Genscript) via Michael-type addition in 0.2 M triethanolamine at pH 8, dialyzed against DI water, and then lyophilized. The final RGD concentration was 1 mM for all hydrogel formulations used for cell culture. After materials were put into solution (non-fibrous MeHA and guest-host hydrogels) or hydrated (guest-host fibers), the materials were sterilized using germicidal UV irradiation for 3 h. Prior to the addition of cells, guest-host fibers were centrifuged briefly, with excess liquid aspirated under sterile conditions. To evaluate cell protection during injection under needle flow, hMSCs were added to the non-fibrous hydrogels or hydrogel fibers such that the final encapsulation density was 1×10⁶ cells/mL. Prior to UV exposure, materials with cells were loaded into 1 mL tuberculin syringes and hydrogels were injected through a 16G needle at 12 mL h⁻¹ using a syringe pump. The extruded material was used to make hydrogel plugs with identical volumes of 40 μL. All groups were subjected to 5 min UV polymerization post-injection to ensure that differences in cell viability were not due to UV cytotoxicity. For all cell culture experiments, media was replaced every 2-3 days.

Confocal imaging and analysis. Cell viability was assessed with a LIVE/DEAD assay kit (Invitrogen) by incubating hydrogels at 37° C. for 30 min with 2 mM calcein-AM and 4 mM ethidium homodimer-1 in PBS plus glucose (G-PBS, glucose 25 mM) followed by three PBS washes. Cells were imaged using a Leica DMi8 inverted confocal microscope and live/dead cells were quantified from 50 μm thick z-stacks (10 μm slices) using ImageJ's 3D object counter tool. Cell projected area, cell shape index (CSI), and aspect ratio were determined using ImageJ's measurement tool. CSI determines the circularity of the cell, where a line and a circle have CSI values of 0 and 1, respectively, and was calculated using the formula

$$C S I = \frac{4 π A}{{P}^{2}}$$

where A is the cell projected area and P is the cell perimeter. Individual measurements were made for 60 cells from at least 6 hydrogels per experimental group.

Statistical Analysis. All experimental groups included at least 6 hydrogels for analysis of cell viability and shape. Cell viability and shape data were statistically analyzed using one-way analysis of variance (ANOVA) followed by Tukey's HSD post-hoc testing. These statistical analyses were conducted using GraphPad Prism 9.0 and R. P values <0.05 were considered statistically significant. For analysis of hydrogel rheological properties, 3 hydrogels were tested per group where the mean values are plotted. Bar graph heights correspond to the mean with standard deviation error bars and individual data points are included as scatter plots overlaying the bars. Tukey box plots of individual cell data show the second and third quartiles as boxes, the median as a line between the boxes, and error bars with the lower value of either 1.5 times the interquartile range or the maximum/minimum value. Data points outside this range are shown individually.

### Example 1

Guest-Host Hydrogel Fiber Design, Synthesis, and Quantification of Fiber Properties.

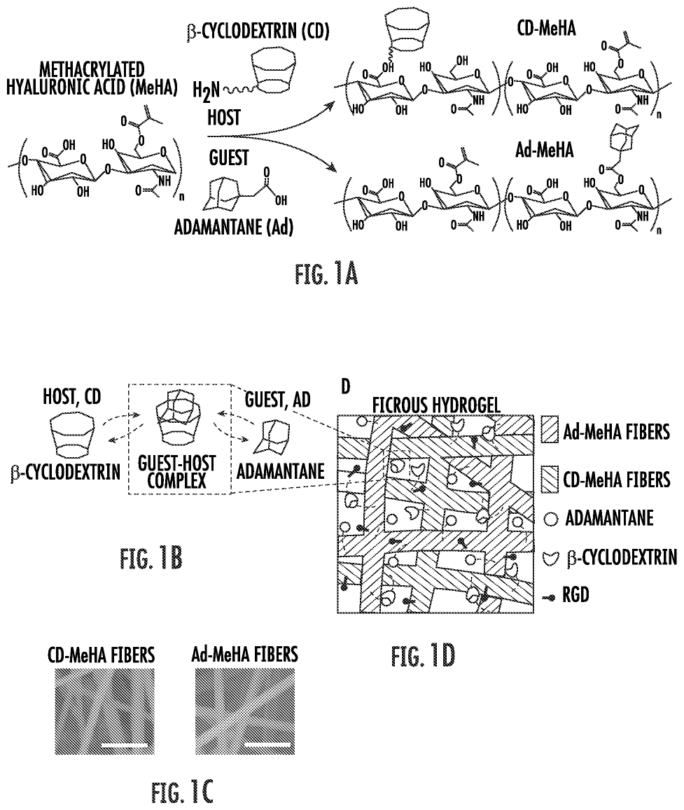

Disclosed herein is the design, synthesis, and characterization of a self-assembling guest-host fibrous hydrogel that can serve as a cell carrier for injectable tissue engineering applications. Hydrophobic Ad (guest) groups associate strongly with the hydrophobic core of CD (host) molecules resulting in a stable supramolecular, yet reversible, guest-host interaction (association constant, Kₐ=1×10⁵ M₋₁) (FIG. 1). Ad and CD moieties were separately coupled to methacrylate-modified HA (MeHA), forming Ad-MeHA and CD-MeHA respectively (FIG. 1A). Modification of the HA backbone with methacrylate groups enables covalent photocrosslinking of the electrospun hydrogel nanofibers in the presence of photoinitiator via UV light-mediated radical polymerization. Following photocrosslinking, the stabilized polymeric fibers will imbibe water upon hydration rather than dissolving, creating a water-swollen hydrogel fiber structure. When the hydrated guest and host fibers are mixed, it was discovered that the complementary nanofibers associate via hydrophobic supramolecular interactions to form a mechanically robust 3D fibrous hydrogel (FIG. 1D) capable of shear-thinning and self-healing. The supramolecularly-assembled fibers can create a hierarchical assembly that provide physical cues to cells at different length scales, mimicking the 3D cues provided by the native fibrous ECM. Additionally, the hydrogel design enables the facile addition of ligands, such as cell adhesion peptides, creating the potential to capture and independently modulate multiple features of native ECM in addition to biophysical cues.

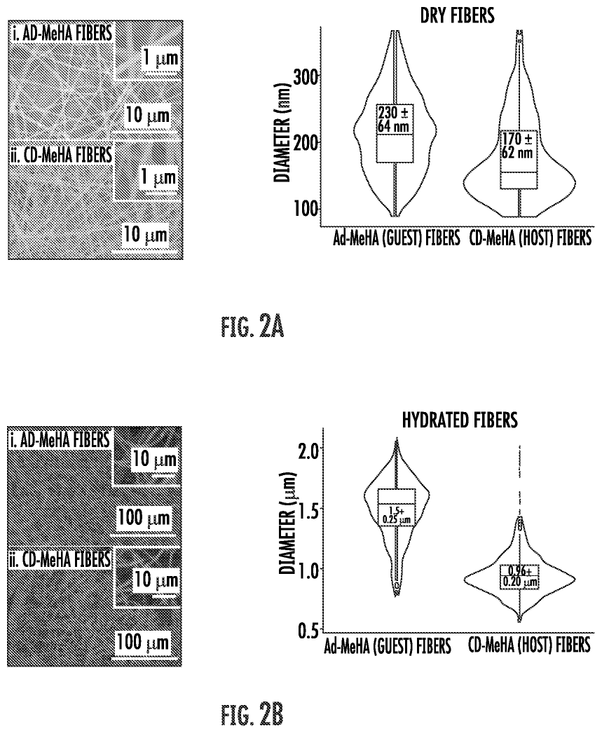

To create the electrospun hydrogel nanofibers capable of supramolecular assembly, Ad-MeHA and CD-MeHA (2% w/v) aqueous polymer solutions were mixed separately with poly(ethylene oxide) (PEO) and the photoinitiator Irgacure 2959 (I2959), and then electrospun to produce guest and host fiber populations (FIG. 1C). Addition of the photoinitiator allowed for subsequent stabilization of the fibers by UV light-mediated radical crosslinking of methacrylates while PEO was included as a bioinert carrier polymer. As a carrier polymer, PEO aids in the electrospinning process by making the solution more viscous, thereby disrupting the relatively high surface tension and inducing chain entanglements of the low molecular weight HA solution⁵². Crosslinked hydrogel nanofibers were examined in their dry state via scanning electron microscopy (SEM, FIG. 2A) and in their hydrated form via confocal microscopy (FIG. 2B). Dry Ad-MeHA nanofibers had an average diameter of 234±64 nm while the average dry CD-MeHA fiber diameter was 171±64 nm.

Upon hydration, the hydrophilic nanofibers imbibe water, resulting in significant fiber swelling and increased diameter. The hydrated Ad-MeHA nanofibers swelled to an average diameter of 2.16±0.92 μm and hydrated CD-MeHA nanofibers swelled to an average diameter of 1.65±0.54 μm. SEM analysis of native fibrous ECMs has reported fibril diameters in the range of 75-400 nm^{57,58} with small type I collagen fibers in the 1-5 μm range⁵⁹. Therefore, these CD-MeHA and Ad-MeHA hydrogel nanofibers are within the physiologically relevant range for fibrous ECM components¹⁵. In addition, both the distribution of hydrogel nanofiber diameters and the morphological change of the nanofibers following hydration (and the resulting PEO extraction) were observed. Calculated swelling ratios for the guest Ad-MeHA fibers and host CD-MeHA fibers were 93±15 and 49±4.8 respectively. These results correlate with the greater degree of swelling in the Ad-MeHA fibers seen in FIG. 2 as quantified by the wet/dry fold change in fiber diameter (about 5.5 for Ad-MeHA versus about 4.6 for CD-MeHA). The extent of swelling for HA fibers can depend on the crosslinking density with denser crosslinking leading to reduced fiber swelling. However, this does not account for the observed differences in fiber swelling between the Ad-MeHA and CD-MeHA fibers since both formulations have the same degree of methacrylate modification. Without being bound by any particular theory or mechanism of action, one possible explanation for the CD-MeHA fibers being smaller is a packing phenomenon previous reported where cyclodextrin can form nanoscale aggregates. Additionally, the hydrophobic nature of the bulky CD group (1.53 nm outer diameter and tapered cavity 0.79 nm in height) coupled with the hydrophobic hexamethylenediamine linker used to conjugate the CD to HA could also influence the reduced swelling in these fibers. Even though the degree of modification with Ad was high (43%), the guest Ad-MeHA fibers were still about 80% hydrophilic HA by mass. In contrast, the CD-MeHA host fibers were only about 55% HA by mass.

### Example 2

Guest-Host-Assembled Fibers Show Mechanical Integrity as Well as Shear-Thinning and Self-Healing Character.

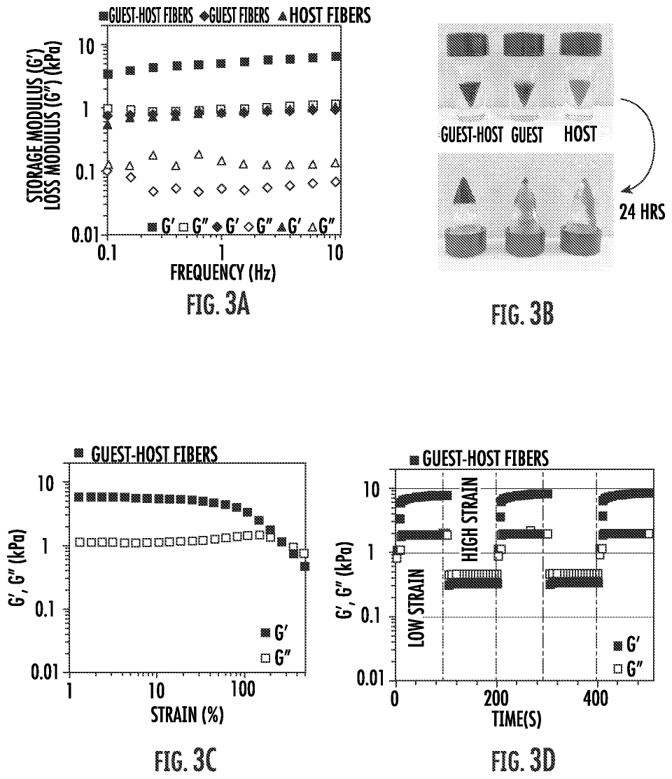

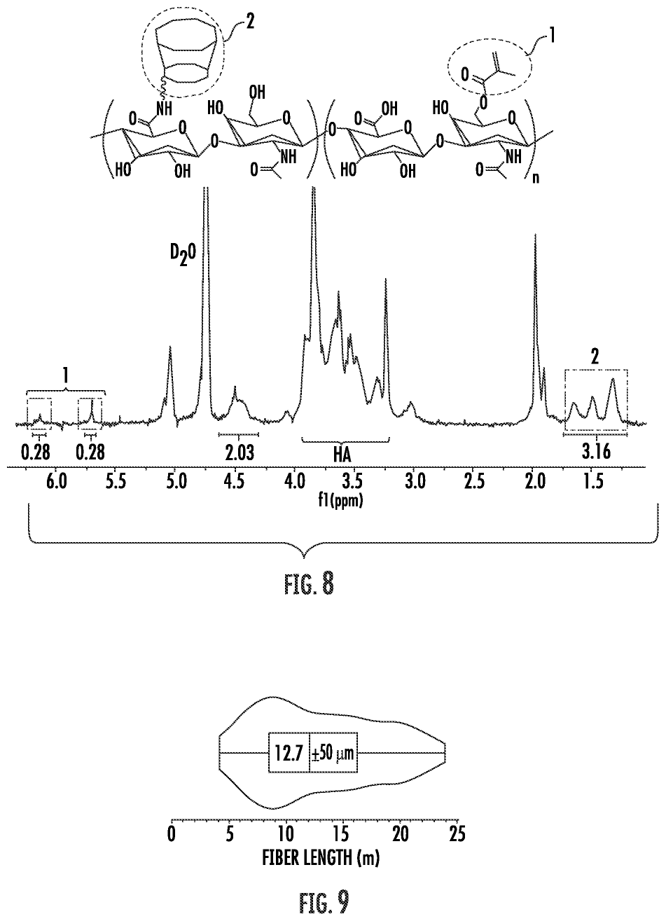

With the stable structure of the hydrated electrospun hydrogel nanofibers confirmed via confocal microscopy, mechanical properties of the fibrous hydrogels were then characterized via oscillatory shear rheology (FIG. 3). To form fibrous hydrogels amenable to injection and cell encapsulation, hydrated hydrogel fibers were triturated repeatedly via needle extrusion to produce short fiber segments of length 12.7±5.0 μm (FIG. 9). A 2:1 molar ratio of guest Ad to host CD groups within the fiber mixture was chosen based on the expectation that excess Ad group can promote longer-term mechanical stability. The fiber density of each group in FIG. 3 was 1% w/v such that any change in material properties could be directly attributed to guest-host interactions. Guest and host fiber populations, measured separately, showed a higher storage modulus than loss modulus as these bulk measurements reflect the properties of the photocrosslinked fibers and their ability to entangle. The guest-host fibrous network also demonstrated a higher storage modulus (G′, 6.6±2.0 kPa) than loss modulus (G″, 1.2±0.5 kPa), but the increase in storage modulus of the mixed guest-host fibers compared to the individual fiber populations highlights the combined mechanical contributions of the individual covalently-crosslinked fibers and the supramolecular interactions between complementary fiber types (FIG. 3A). Importantly, the guest-host interactions between complementary fibers were necessary for longer-term mechanical stability as shown by a qualitative vial inversion test (FIG. 3B).

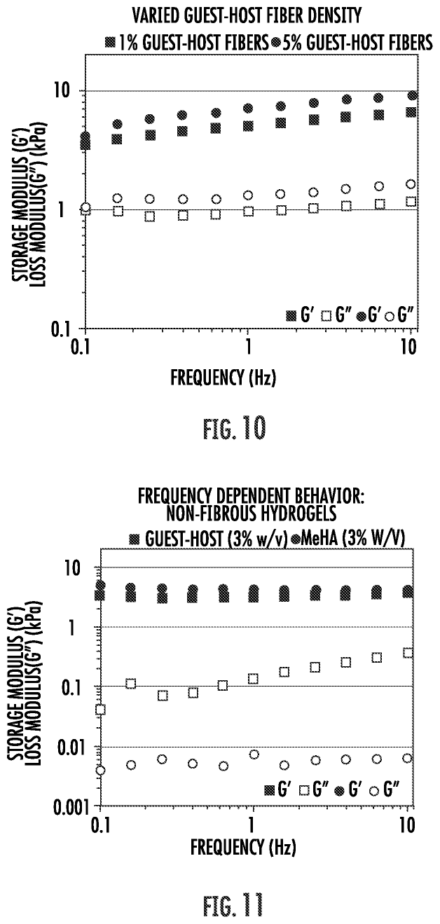

Biopolymer density can also be adjusted to tune mechanical properties. Fibrous hydrogel matrices formed with 5% fiber density (also 2:1 Ad:CD molar ratio) showed improved storage moduli over the 1% fiber density formulation (FIG. 10). The results of these studies found that the guest-host pair outperformed groups without supramolecular assembly in terms of adhesion strength between the fiber layers. Without being bound by any particular theory or mechanism of action, in some embodiments it may be that smaller fiber diameters can lead to increased mechanical properties because the increased surface area to volume ratio would result in higher surface availability of Ad and CD groups to associate with each other. The ability to tune the mechanical properties of the fibrous hydrogel scaffold, by controlling the density of fibers and the ratio of host to guest moieties, is an important feature that may allow access to a range of mechanical properties suitable for fibrous tissue repair.

Having demonstrated the guest-host assembly of the functionalized HA hydrogel fibers, efforts were next taken to assess their shear-thinning and self-healing capabilities. Strain sweep testing of the fibrous guest-host hydrogel showed loss moduli crossover at strains greater than 100%, highlighting the potential for injectability (FIG. 3C). The guest-host fibrous scaffold was then subjected to cyclic deformations to investigate the responsiveness to shear force and the ability of the fibers to undergo self-healing, i.e., testing the injectable capabilities of the material. Shear-thinning hydrogels will demonstrate sharp decreases in storage and loss moduli when subjected to shear forces with loss moduli crossover (exceeding storage moduli), which is predictive of liquid-like behavior during needle flow upon injection. Conversely, upon cessation of shear, the storage and loss moduli recover for self-healing hydrogels. Multiple cycles of high strain (250%) followed by short recovery periods at low strain (0.5% strain) demonstrated the guest-host fibrous hydrogel's ability to both shear-thin and self-heal following mechanical deformation (FIG. 3D). Based on these experimental findings, the shear forces associated with syringe-based extrusion would induce the same reversible liquid-like to solid-like transitions.

Studies using non-fibrous Ad-CD guest-host assembled hydrogels demonstrated the tunable mechanics and flow characteristics of the guest-host assembly by altering the guest-host pair ratio and density as well as network structure^{42,72,73}. The fibrous hydrogel developed and disclosed herein displays analogous rheological behavior to non-fibrous guest-host hydrogels. However, a surprising advantage over the non-fibrous hydrogels is that as a result of the dynamic bonding interactions between fibers, the guest-host fibrous hydrogel is capable of shear-induced flow (injectability) and rapid recovery. Overall, rheological analysis of the guest-host fibrous hydrogel demonstrated properties of robust mechanical integrity, shear-thinning, and rapid recovery for stability post-injection. Indeed, the fibrous hydrogel scaffold was readily injectable, flowing easily through a needle (12 mL h⁻¹, 16G) and recovered as a stable hydrogel plug.

### Example 3

Injected hMSCs Encapsulated in Fibrous Hydrogels are Viable and Show Increased Spreading Compared to Non-Fibrous Hydrogels.

Experiments were next designed to test the ability of the fibrous guest-host hydrogel to support sustained viability and spreading of encapsulated hMSCs following injection. hMSCs were chosen for these experiments due to their multipotential for differentiation toward cell types relevant for a broad range of fibrous tissues⁷⁴ such as muscle, tendon, and ligament⁷⁵. Protecting cells during injection and preserving high viability is one of the fundamental requirements for subsequent therapeutic application, but many injectable delivery vehicles suffer from poor cell survival^{76,77}. Previous studies have attempted to address this issue using materials that leverage physical crosslinking since their compliant mechanical properties support non-uniform network deformation^{40,68,78}. However, in these non-uniform matrices, mechanical stresses imposed on the material do not induce uniform strain fields allowing network deformations to dissipate stress⁸. Therefore, there remained a need to address this deficiency, which led to the design and discovery that the disclosed guest-host fibers could mitigate cell-damaging forces during injection due to the force dissipation capabilities of shear-thinning materials.

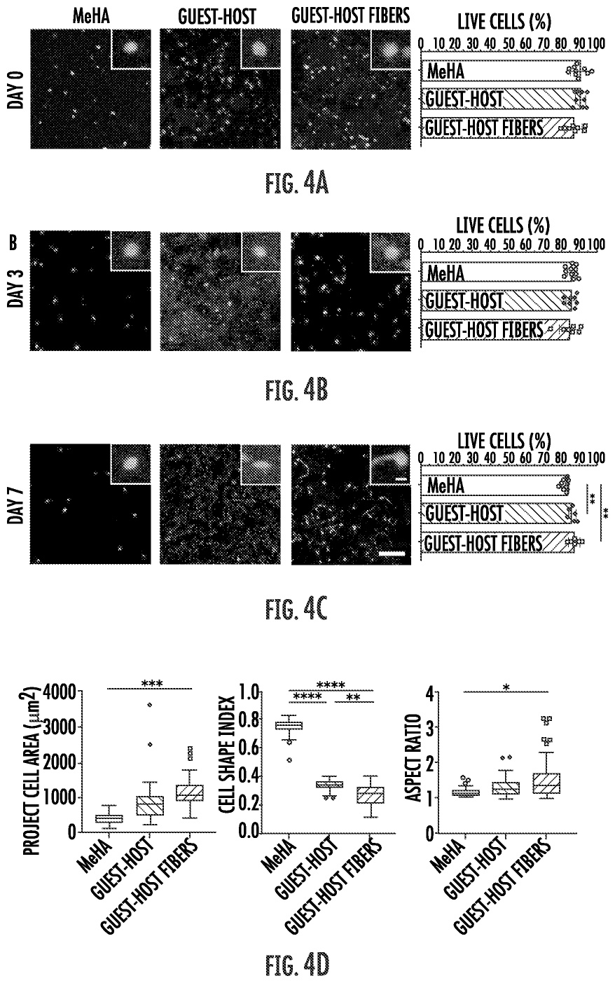

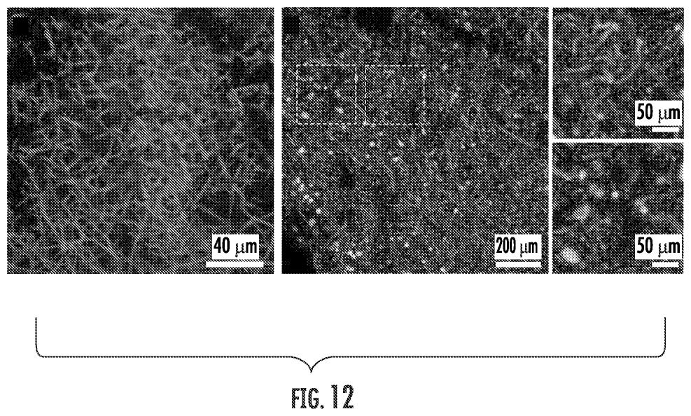

The post-injection viability and spreading of hMSCs when cultured in three hydrogel groups: non-fibrous MeHA, non-fibrous guest-host, and fibrous guest-host (FIG. 4) was investigated. Groups were chosen such that the influence of fibrous architecture on cell behavior could be assessed independently from the role of guest-host complexation. All groups contained 1 mM RGD peptide to support cell adhesion and had similar storage moduli (rheology of non-fibrous hydrogels is shown in FIG. 11). Cell viability was quantified at three time points: immediately following injection, day 3 of culture, and day 7 of culture. Following injection, hMSCs in the fibrous guest-host hydrogel were 85±5% viable, a value that was statistically indistinguishable from the hMSCs encapsulated in either of the non-fibrous hydrogels: MeHA (88±4%) or guest-host (89±3%). Previous studies have reported the highest cell survival in injectable hydrogels (about 90%) utilizing formulations with modest mechanical properties (G′ about 30 Pa)^{76,79}. More recently, a self-assembled fibrous peptide hydrogel reported 86.8% cell viability post-injection despite a substantially higher storage modulus (3.1 kPa)⁴⁰. One possible explanation for the fibrous materials' ability to protect cells during injection, despite the more robust bulk mechanical stiffnesses compared to other successful cell carrier materials, is the stochastic nature of self-assembling fiber hierarchical structures allowing microstructural deformation mechanisms such as shear attenuation via fiber sliding. Local shear attenuation can be important for native tissue mechanical function and may be mimicked by the supramolecular interactions between complementary guest and host hydrogel fibers, leading to increased force dissipation and thereby protecting encapsulated cells from extensional flow at the entrance of the syringe needle and the subsequent disruption of the cellular membrane.

Similar to the initial cell viability, the live cell fractions observed on days 3 and 7 were largely the same (day 3: non-fibrous MeHA: 83±3% and guest-host: 83±3% versus the fibrous guest-host hydrogel: 82±6%), although on day 7 there was a statistically significant difference in viability between the non-fibrous MeHA hydrogel group compared to both guest-host hydrogel groups (non-fibrous: 86±2%, fibrous: 87±3%). Although hMSC viability was similar across all experimental groups, qualitative differences in cell shape/spreading observed at day 7 provided motivation to quantify hMSC shape metrics such as projected cell area, cell shape index (CSI, a measure of cell circularity), and aspect ratio (FIG. 4D). The ability of encapsulated hMSCs to spread is particularly important for supporting the active mechanical signaling⁸³ necessary for cells to differentiate⁸⁴ and deposit tissue-specific ECM^{85,86} during fibrous musculoskeletal tissue repair.

Confocal imaging of hMSCs in fibrous networks showed direct hMSC interactions with fiber segments (FIG. 12) as well as the highest levels of spreading and elongation (as measured by both CSI and aspect ratio) compared to the non-fibrous hydrogel groups. In particular, the differences in cell shape were the greatest between cells encapsulated in the guest-host hydrogels, both fibrous (spread area: 1130±146 μm², CSI: 0.28±0.04, aspect ratio: 1.54±0.33) and non-fibrous (spread area: 850±333 μm², CSI: 0.34±0.02, aspect ratio: 1.33±0.09) compared to the MeHA hydrogels which showed more rounded hMSC morphologies (spread area: 400±93 μm², CSI: 0.76±0.02, aspect ratio: 1.17±0.05). The reduction in hMSC spreading and elongation found in MeHA hydrogels compared to the guest-host hydrogels is likely due to differences in viscoelasticity. The guest-host hydrogel networks contain both covalent crosslinking and guest-host supramolecular interactions, leading to viscoelastic properties as shown in the disclosed rheological analysis while MeHA hydrogels are covalently crosslinked and behave like elastic solids. These findings show a significant reduction in CSI observed for hMSCs in fibrous guest-host hydrogels compared to non-fibrous guest host hydrogels. Without being bound by any particular theory or mechanism of action, possible explanations for these observed results include CD interactions with aromatic amino acids to provide additional cell anchoring points and local strain stiffening via fiber recruitment as observed in native ECM and in hydrogel fiber networks. Together, the data from these experiments underscore the advantageous suitability of the disclosed fibrous hydrogel design for injectable encapsulation of hMSCs in a microenvironment supportive of cell spreading and elongation.

### Discussion of Examples 1-3

In summary, the present disclosure details the design and fabrication of an injectable fibrous hydrogel providing significant advantages over existing hydrogels, including for example, the capabilities of shear-thinning and self-healing under physiologic conditions. Using HA as a naturally-derived polymer, these materials offer unique advantages with respect to biocompatibility and mechanical integrity (G′=6.6±2.0 kPa) while also recapitulating the fibrous architecture of tissue. In addition to exhibiting shear-thinning and self-healing behavior, the guest-host fibrous hydrogel demonstrated injectability wherein encapsulated hMSCs were protected from membrane-disrupting shear forces during injection, resulting in sustained 3D hMSC viability (greater than 85%). Cells were also more spread and elongated in viscoelastic guest-host hydrogels compared to non-fibrous elastic hydrogel controls, with hMSCs showing significantly reduced CSI (circularity) in fibrous guest-host hydrogels compared to both non-fibrous MeHA and non-fibrous guest-host hydrogels. In summary, the injectable fibrous hydrogel platform introduced here offers the ability to broaden minimally invasive delivery to musculoskeletal tissue engineering applications requiring robust structural properties while also laying the foundation for future opportunities in 3D bioprinting and fundamental studies of cell-microenvironment interactions.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
(1) Frantz, C.; Stewart, K. M.; Weaver, V. M. The Extracellular Matrix at a Glance. J. Cell Sci. 2010, 123, 4195-4200. https://doi.org/10.1242/jcs. 023820.
(2) Rosso, F.; Giordano, A.; Barbarisi, M.; Barbarisi, A. From Cell-ECM Interactions to Tissue Engineering. J. Cell. Physiol. 2004, 199 (2), 174-180. https://doi.org/10.1002/jcp.10471.
(3) Lee, K. Y.; Mooney, D. J. Hydrogels for Tissue Engineering. Chem. Rev. 2001, 101 (7), 1869-1879. https://doi.org/10.1021/cr000108x.
(4) Courtney, T.; Sacks, M. S.; Stankus, J.; Guan, J.; Wagner, W. R. Design and Analysis of Tissue Engineering Scaffolds That Mimic Soft Tissue Mechanical Anisotropy. Biomaterials 2006, 27 (19), 3631-3638. https://doi org/10.1016/j.biomaterials 0.2006.02.024.
(5) Caliari, S. R.; Burdick, J. A. A Practical Guide to Hydrogels for Cell Culture. Nat. Methods 2016, 13 (5), 405-414. https://doi.org/10.1038/nmeth.3839.
(6) Vasita, R.; Katti, D. S. Nanofibers and Their Applications in Tissue Engineering. Int. J. Nanomedicine 2006, 1 (1), 15-30. https://doi.org/10.2147/nano.2006.1.1.15.
(7) Pathak, A.; Kumar, S. Biophysical Regulation of Tumor Cell Invasion: Moving beyond Matrix Stiffness. Integr. Biol. 2011, 3 (4), 267-278. https://doi.org/10.1039/c0ib00095g.
(8) Pedersen, J. A.; Swartz, M. A. Mechanobiology in the Third Dimension. Ann. Biomed. Eng. 2005, 33 (11), 1469-1490. https://doi.org/10.1007/s10439-005-8159-4.
(9) Rose, J. C.; De Laporte, L. Hierarchical Design of Tissue Regenerative Constructs. Adv. Healthc. Mater. 2018, 7 (6), 1-31. https://doi.org/10.1002/adhm.201701067.
(10) Alberts, B.; Johnson, A.; Lewis, J.; Raff, M.; Roberts, K. Molecular Biology of the Cell, 4th Edition; Garland Science: New York, NY, 2002.
(11) Burdick, J. A.; Murphy, W. L. Moving from Static to Dynamic Complexity in Hydrogel Design. Nat. Commun. 2012, 3. https://doi.org/10.1038/ncomms2271.
(12) Hinderer, S.; Layland, S. L.; Schenke-Layland, K. ECM and ECM-like Materials-Biomaterials for Applications in Regenerative Medicine and Cancer Therapy. Adv. Drug Deliv. Rev. 2016, 97, 260-269. https://doi.org/10.1016/j.addr.2015.11.019.
(13) Tibbitt, M. W.; Anseth, K. S. Hydrogels as Extracellular Matrix Mimics for 3D Cell Culture. Biotechnol. Bioeng. 2009, 103 (4), 655-663. https://doi.org/10.1002/bit.22361.
(14) Fu, Q.; Duan, C.; Yan, Z.; Li, Y.; Si, Y.; Liu, L.; Yu, J.; Ding, B. Nanofiber-Based Hydrogels: Controllable Synthesis and Multifunctional Applications. Macromol. Rapid Commun. 2018, 39 (10), 1-19. https://doi.org/10.1002/marc.201800058.
(15) Greiner, A.; Wendorff, J. H. Electrospinning: A Fascinating Method for the Preparation of Ultrathin Fibers. Angew Chem Int Ed 2007, 46 (30), 5670-5703. https://doi.org/10.1002/anie.200604646.
(16) Li, W.-J.; Mauck, R. L.; Tuan, R. S. Electrospun Nanofibrous Scaffolds: Production, Characterization, and Applications for Tissue Engineering and Drug Delivery. J. Biomed. Nanotechnol. 2006, 1 (3), 259-275. https://doi.org/10.1166/jbn.2005.032.
(17) Teo, W. E.; Ramakrishna, S. A Review on Electrospinning Design and Nanofibre Assemblies. Nanotechnology 2006, 17 (14). https://doi.org/10.1088/0957-4484/17/14/R01.
(18) Christopherson, G. T.; Song, H.; Mao, H. Q. The Influence of Fiber Diameter of Electrospun Substrates on Neural Stem Cell Differentiation and Proliferation. Biomaterials 2009, 30 (4), 556-564. https://doi org/10.1016/j.biomaterials 0.2008.10.004.
(19) Grewal, M. G.; Highley, C. B. Electrospun Hydrogels for Dynamic Culture Systems: Advantages, Progress, and Opportunities. Biomater. Sci. 2021. https://doi.org/10.1039/d0bm01588a.
(20) Lutolf, M. P.; Hubbell, J. A. Synthetic Biomaterials as Instructive Extracellular Microenvironments for Morphogenesis in Tissue Engineering. Nat. Biotechnol. 2005, 23 (1), 47-55. https://doi.org/10.1038/nbt1055.
(21) Zhang, X.; Shi, X.; Gautrot, J. E.; Peijs, T. Nanoengineered Electrospun Fibers and Their Biomedical Applications: A Review. Nanocomposites 2020, 7 (1), 1-34. https://doi.org/10.1080/20550324.2020.1857121.
(22) Burdick, J. A.; Prestwich, G. D. Hyaluronic Acid Hydrogels for Biomedical Applications. Adv. Mater. 2011, 23 (12), 41-56. https://doi.org/10.1002/adma.201003963.
(23) Highley, C. B.; Prestwich, G. D.; Burdick, J. A. Recent Advances in Hyaluronic Acid Hydrogels for Biomedical Applications. Curr. Opin. Biotechnol. 2016, 40, 35-40. https://doi.org/10.1016/j.copbio.2016.02.008.
(24) Scott, J. E. Extracellular Matrix, Supramolecular Organisation and Shape. J. Anat. 1995, 187, 259-269. https://doi.org/10.1016/0277-9536(83)90357-x.
(25) Baker, B. M.; Trappmann, B.; Wang, W. Y.; Sakar, M. S.; Kim, I. L.; Shenoy, V. B.; Burdick, J. A.; Chen, C. S. Cell-Mediated Fibre Recruitment Drives Extracellular Matrix Mechanosensing in Engineered Fibrillar Microenvironments. Nat. Mater. 2015, 14 (12), 1262-1268. https://doi.org/10.1038/nmat4444.
(26) Xie, J.; Bao, M.; Bruekers, S. M. C.; Huck, W. T. S. Collagen Gels with Different Fibrillar Microarchitectures Elicit Different Cellular Responses. ACS Appl. Mater. Interfaces 2017, 9 (23), 19630-19637. https://doi.org/10.1021/acsami.7b03883.
(27) Baker, B. M.; Chen, C. S. Deconstructing the Third Dimension-How 3D Culture Microenvironments Alter Cellular Cues. J. Cell Sci. 2012, 125 (13), 3015-3024. https://doi.org/10.1242/jcs.079509.
(28) Matera, D. L.; Wang, W. Y.; Smith, M. R.; Shikanov, A.; Baker, B. M. Fiber Density Modulates Cell Spreading in 3D Interstitial Matrix Mimetics. ACS Biomater. Sci. Eng. 2019, 5 (6), 2965-2975. https://doi.org/10.1021/acsbiomaterials.9b00141.
(29) Hilderbrand, A. M.; Ford, E. M.; Guo, C.; Sloppy, J. D.; Kloxin, A. M. Hierarchically Structured Hydrogels Utilizing Multifunctional Assembling Peptides for 3D Cell Culture. Biomater. Sci. 2020, 8 (5), 1256-1269. https://doi.org/10.1039/c9bm01894h.
(30) Matellan, C.; del Rio Hernandez, A. E. Engineering the Cellular Mechanical Microenvironment-From Bulk Mechanics to the Nanoscale. J. Cell Sci. 2019, 132 (9). https://doi.org/10.1242/jcs.229013.
(31) Li, J.; Mooney, D. J. Designing Hydrogels for Controlled Drug Delivery. Nat. Rev. Mater. 2016. https://doi.org/10.1097/IOP.0b013e31819ac7c5.
(32) Dimatteo, R.; Darling, N. J.; Segura, T. In Situ Forming Injectable Hydrogels for Drug Delivery and Wound Repair. Adv. Drug Deliv. Rev. 2018, 127, 167-184. https://doi.org/10.1016/j.addr.2018.03.007.
(33) Jammalamadaka, U.; Tappa, K. Recent Advances in Biomaterials for 3D Printing and Tissue Engineering. J. Funct. Biomater. 2018, 9 (1). https://doi.org/10.3390/jfb9010022.
(34) Loebel, C.; Rodell, C. B.; Chen, M. H.; Burdick, J. A. Shear-Thinning and Self-Healing Hydrogels as Injectable Therapeutics and for 3D-Printing. Nat. Protoc. 2017, 12 (8), 1521-1541. https://doi.org/10.1038/nprot.2017.053.
(35) Yu, L.; Ding, J. Injectable Hydrogels as Unique Biomedical Materials. Chem Soc Rev 2008, 1473-1481. https://doi.org/10.1039/b713009k.
(36) Park, H.; Woo, E. K.; Lee, K. Y. Ionically Cross-Linkable Hyaluronate-Based Hydrogels for Injectable Cell Delivery. J Controlled Release 2014, 196, 146-153. https://doi.org/10.1016/j.jconre1.2014.10.008.
(37) Highley, C. B.; Song, K. H.; Daly, A. C.; Burdick, J. A. Jammed Microgel Inks for 3D Printing Applications. Adv. Sci. 2019, 6 (1). https://doi.org/10.1002/advs.201801076.
(38) Riley, L.; Schirmer, L.; Segura, T. Granular Hydrogels: Emergent Properties of Jammed Hydrogel Microparticles and Their Applications in Tissue Repair and Regeneration. Curr. Opin. Biotechnol. 2019, 60, 1-8. https://doi.org/10.1016/j.copbio.2018.11.001.
(39) Mann, J. L.; Yu, A. C.; Agmon, G.; Appel, E. Supramolecular Polymeric Biomaterials. Biomater. Sci. 2017. https://doi.org/10.1039/C7BM00780A.
(40) Tang, J. D.; Roloson, E. B.; Amelung, C. D.; Lampe, K. J. Rapidly Assembling Pentapeptides for Injectable Delivery (RAPID) Hydrogels as Cytoprotective Cell Carriers. ACS Biomater. Sci. Eng. 2019, 5 (5), 2117-2121. https://doi.org/10.1021/acsbiomaterials.9b00389.
(41) Hu, C.; Zhang, F.; Long, L.; Kong, Q.; Luo, R.; Wang, Y. Dual-Responsive Injectable Hydrogels Encapsulating Drug-Loaded Micelles for on-Demand Antimicrobial Activity and Accelerated Wound Healing. J. Controlled Release 2020, 324 (May), 204-217. https://doi.org/10.1016/j.jconre1.2020.05.010.
(42) Rodell, C. B.; Kaminski, A. L.; Burdick, J. A. Rational Design of Network Properties in Guest-Host Assembled and Shear-Thinning Hyaluronic Acid Hydrogels. Biomacromolecules 2013, 14 (11), 4125-4134. https://doi.org/10.1021/bm401280z.
(43) Rodell, C. B.; MacArthur, J. W.; Dorsey, S. M.; Wade, R. J.; Wang, L. L.; Woo, Y. J.; Burdick, J. A. Shear-Thinning Supramolecular Hydrogels with Secondary Autonomous Covalent Crosslinking to Modulate Viscoelastic Properties in Vivo. Adv. Funct. Mater. 2015, 25 (4), 636-644. https://doi.org/10.1002/adfm.201403550.
(44) Gaffey, A. C.; Chen, M. H.; Venkataraman, C. M.; Trubelja, A.; Rodell, C. B.; Dinh, P. V.; Hung, G.; MacArthur, J. W.; Soopan, R. V.; Burdick, J. A.; Atluri, P. Injectable Shear-Thinning Hydrogels Used to Deliver Endothelial Progenitor Cells, Enhance Cell Engraftment, and Improve Ischemic Myocardium. J. Thorac. Cardiovasc. Surg. 2015, 150 (5), 1268-1277. https://doi.org/10.1016/j.jtcvs.2015.07.035.
(45) Rajagopal, K.; Schneider, J. P. Self-Assembling Peptides and Proteins for Nanotechnological Applications. Curr. Opin. Struct. Biol. 2004, 14 (4), 480-486. https://doi.org/10.1016/j.sbi.2004.06.006.
(46) Khorshidi, S.; Solouk, A.; Mirzadeh, H.; Mazinani, S.; Lagaron, J. M.; Sharifi, S.; Ramakrishna, S. A Review of Key Challenges of Electrospun Scaffolds for Tissue-Engineering Applications. J Tissue Eng Regen Med 2016, 10, 715-738. https://doi.org/10.1002/term.
(47) Wade, R. J.; Burdick, J. A. Engineering ECM Signals into Biomaterials. Mater. Today 2012, 15 (10), 454-459. https://doi.org/10.1016/S1369-7021(12)70197-9.
(48) Smeds, K. A.; Grinstaff, M. W. Photocrosslinkable Polysaccharides for in Situ Hydrogel Formation. J Biomed. Mater. Res. 2001, 54 (1), 115-121. https://doi.org/10.1002/1097-4636(200101)54:1<HS::AID-JBM14>3.0. CO; 2-Q.
(49) Highley, C. B.; Rodell, C. B.; Burdick, J. A.; Highley, C. B.; Rodell, C. B.; Burdick, J. A. Direct 3D Printing of Shear-Thinning Hydrogels into Self-Healing Hydrogels. Adv. Mater. 2015, 27, 5057-5079. https://doi.org/10.1002/adma.201501234.
(50) Sundararaghavan, H. G.; Burdick, J. A. Gradients with Depth in Electrospun Fibrous Scaffolds for Directed Cell Behavior. Biomacromolecules 2011, 12 (6), 2344-2350. https://doi.org/10.1021/bm200415g.
(51) Highley, C. B.; Rodell, C. B.; Kim, I. L.; Wade, R. J.; Burdick, J. A. Ordered, Adherent Layers of Nanofibers Enabled by Supramolecular Interactions. J Mater Chem B 2014, 2, 8110-8115. https://doi.org/10.1039/c4tb00724g.
(52) Ji, Y.; Ghosh, K.; Shu, X. Z.; Li, B.; Sokolov, J. C.; Prestwich, G. D.; Clark, R. A. F.; Rafailovich, M. H. Electrospun Three-Dimensional Hyaluronic Acid Nanofibrous Scaffolds. Biomaterials 2006, 27 (20), 3782-3792. https://doi.org/10.1016/j.biomaterials.2006.02.037.
(53) Fairbanks, B. D.; Schwartz, M. P.; Bowman, C. N.; Anseth, K. S. Photoinitiated Polymerization of PEG-Diacrylate with Lithium Phenyl-2,4,6-Trimethylbenzoylphosphinate: Polymerization Rate and Cytocompatibility. Biomaterials 2009, 30 (35), 6702-6707. https://doi org/10.1016/j.biomaterials 0.2009.08.055.
(54) Chen, G.; Jiang, M. Cyclodextrin-Based Inclusion Complexation Bridging Supramolecular Chemistry and Macromolecular Self-Assembly. Chem. Soc. Rev. 2011, 40 (5), 2254-2266. https://doi.org/10.1039/c0cs00153h.
(55) Burdick, J. A.; Chung, C.; Jia, X.; Randolph, M. A.; Langer, R. Controlled Degradation and Mechanical Behavior of Photopolymerized Hyaluronic Acid Networks. Biomacromolecules 2005, 6 (1), 386-391. https://doi.org/10.1021/bm049508a.
(56) Wade, R. J.; Bassin, E. J.; Gramlich, W. M.; Burdick, J. A. Nanofibrous Hydrogels with Spatially Patterned Biochemical Signals to Control Cell Behavior. Adv. Mater. 2015, 27 (8), 1356-1362. https://doi.org/10.1002/adma.201404993.
(57) Abrams, G. A.; Goodman, S. L.; Nealey, P. F.; Franco, M.; Murphy, C. J. Nanoscale Topography of the Basement Membrane Underlying the Corneal Epithelium of the Rhesus Macaque. Cell Tissue Res. 2000, 299 (1), 39-46. https://doi.org/10.1007/s004410050004.
(58) Bancelin, S.; Aimé, C.; Gusachenko, I.; Kowalczuk, L.; Latour, G.; Coradin, T.; Schanne-Klein, M.-C. Determination of Collagen Fibril Size via Absolute Measurements of Second-Harmonic Generation Signals. Nat. Commun. 2014, 5 (1), 4920. https://doi.org/10.1038/ncomms5920.
(59) Ushiki, T. Collagen Fibers, Reticular Fibers and Elastic Fibers. A Comprehensive Understanding from a Morphological Viewpoint. Arch. Histol. Cytol. 2002, 65 (2), 109-126. https://doi.org/10.1679/aohc.65.109.
(60) Ik, S. L.; Oh, H. K.; Meng, W.; Kang, I. K.; Ito, Y. Nanofabrication of Microbial Polyester by Electrospinning Promotes Cell Attachment. Macromol. Res. 2004, 12 (4), 374-378. https://doi.org/10.1007/bf03218414.
(61) Tan, A. R.; Ifkovits, J. L.; Baker, B. M.; Brey, D. M.; Mauck, R. L.; Burdick, J. A. Electrospinning of Photocrosslinked and Degradable Fibrous Scaffolds. J. Biomed. Mater. Res.-Part A 2008, 87 (4), 1034-1043. https://doi.org/10.1002/jbm.a.31853.
(62) Davidson, M. D.; Song, K. H.; Lee, M. H.; Llewellyn, J.; Du, Y.; Baker, B. M.; Wells, R. G.; Burdick, J. A. Engineered Fibrous Networks to Investigate the Influence of Fiber Mechanics on Myofibroblast Differentiation. ACS Biomater. Sci. Eng. 2019, 5 (8), 3899-3908. https://doi.org/10.1021/acsbiomaterials.8b01276.
(63) Wade, R. J.; Bassin, E. J.; Rodell, C. B.; Burdick, J. A. Protease-Degradable Electrospun Fibrous Hydrogels. Nat. Commun. 2015, 6, 6639. https://doi.org/10.1038/ncomms7639.
(64) Kim, I. L.; Khetan, S.; Baker, B. M.; Chen, C. S.; Burdick, J. A. Fibrous Hyaluronic Acid Hydrogels That Direct MSC Chondrogenesis through Mechanical and Adhesive Cues. Biomaterials 2013, 34 (22), 5571-5580. https://doi.org/10.1016/j.biomaterials 0.2013.04.004.
(65) Loftsson, T.; Saokham, P.; Sa. Couto, A. R. Self-Association of Cyclodextrins and Cyclodextrin Complexes in Aqueous Solutions. Int. J. Pharm. 2019, 560 (January), 228-234. https://doi.org/10.1016/j.ijpharm.2019.02.004.
(66) Messner, M.; Kurkov, S. V.; Jansook, P.; Loftsson, T. Self-Assembled Cyclodextrin Aggregates and Nanoparticles. Int. J. Pharm. 2010, 387 (1-2), 199-208. https://doi.org/10.1016/j.ijpharm.2009.11.035.
(67) Zhang, J.; Ma, P. X. Cyclodextrin-Based Supramolecular Systems for Drug Delivery: Recent Progress and Future Perspective. Adv. Drug Deliv. Rev. 2013, 65 (9), 1215-1233. https://doi.org/10.1016/j.addr.2013.05.001.
(68) Sisso, A. M.; Boit, M. O.; DeForest, C. A. Self-Healing Injectable Gelatin Hydrogels for Localized Therapeutic Cell Delivery. J. Biomed. Mater. Res.-Part A 2020, 108 (5), 1112-1121. https://doi.org/10.1002/jbm.a.36886.
(69) Ju, G.; Cheng, M.; Guo, F.; Zhang, Q.; Shi, F. Elasticity-Dependent Fast Underwater Adhesion Demonstrated by Macroscopic Supramolecular Assembly. Angew. Chem.-Int. Ed. 2018, 57 (29), 8963-8967. https://doi.org/10.1002/anie.201803632.
(70) Ju, G.; Cheng, M.; Zhang, Q.; Guo, F.; Xie, P.; Shi, F. Influence of the Surface Chemistry and Dynamics on an Elasticity-Dependent Macroscopic Supramolecular Assembly. ACS Appl. Nano Mater. 2018, 1 (10), 5662-5672. https://doi.org/10.1021/acsanm.8b01277.
(71) Chen, M. H.; Wang, L. L.; Chung, J. J.; Kim, Y. H.; Atluri, P.; Burdick, J. A. Methods to Assess Shear-Thinning Hydrogels for Application As Injectable Biomaterials. ACS Biomater. Sci. Eng. 2017, 3 (12), 3146-3160. https://doi.org/10.1021/acsbiomaterials.7b00734.
(72) Kakuta, T.; Takashima, Y.; Harada, A. Highly Elastic Supramolecular Hydrogels Using Host—Guest Inclusion Complexes with Cyclodextrins. Macromolecules 2013, 46 (11), 4575-4579. https://doi.org/10.1021/ma400695p.
(73) Osman, S. K.; Brandi, F. P.; Zayed, G. M.; TeBmar, J. K.; Gopferich, A. M. Cyclodextrin Based Hydrogels: Inclusion Complex Formation and Micellization of Adamantane and Cholesterol Grafted Polymers. Polymer 2011, 52 (21), 4806-4812. https://doi.org/10.1016/j.polymer.2011.07.059.
(74) Gonzalez-Fernandez, T.; Sikorski, P.; Leach, J. K. Bio-Instructive Materials for Musculoskeletal Regeneration. Acta Biomater. 2019, 96, 20-34. https://doi.org/10.1016/j.actbio.2019.07.014.
(75) Eder, C.; Schmidt-Bleek, K.; Geissler, S.; Sass, F. A.; Maleitzke, T.; Pumberger, M.; Perka, C.; Duda, G. N.; Winkler, T. Mesenchymal Stromal Cell and Bone Marrow Concentrate Therapies for Musculoskeletal Indications: A Concise Review of Current Literature. Mol. Biol. Rep. 2020, 47 (6), 4789-4814. https://doi.org/10.1007/s11033-020-05428-0.
(76) Aguado, B. A.; Mulyasasmita, W.; Su, J.; Ph, D.; Lampe, K. J.; Ph, D.; Heilshom, S. C.; Ph, D. Improving Viability of Stem Cells During Syringe Needle Flow Through the Design of Hydrogel Cell Carriers. Tissue Eng. Part A 2012, 18, 806-815. https://doi.org/10.1089/ten.tea.2011.0391.
(77) Ning, L.; Gil, C. J.; Hwang, B.; Theus, A. S.; Perez, L.; Tomov, M. L.; Bauser-Heaton, H.; Serpooshan, V. Biomechanical Factors in Three-Dimensional Tissue Bioprinting. Appl. Phys. Rev. 2020, 7 (4). https://doi.org/10.1063/5.0023206.
(78) Madl, A. C.; Madl, C. M.; Myung, D. Injectable Cucurbit[8]Uril-Based Supramolecular Gelatin Hydrogels for Cell Encapsulation. ACS Macro Lett. 2020, 9 (4), 619-626. https://doi.org/10.1021/a csmacrolett.0c00184.
(79) Chopin-Doroteo, M.; Mandujano-Tinoco, E. A.; Krötzsch, E. Tailoring of the Rheological Properties of Bioinks to Improve Bioprinting and Bioassembly for Tissue Replacement. Biochim. Biophys. Acta-Gen. Subj. 2021, 1865 (2). https://doi.org/10.1016/j.bbagen.2020.129782.
(80) Knapp, D. M.; Barocas, V. H.; Moon, A. G.; Yoo, K.; Petzold, L. R.; Tranquillo, R. T. Rheology of Reconstituted Type I Collagen Gel in Confined Compression. J. Rheol. 1997, 41 (5), 971-993. https://doi.org/10.1122/1.550817.
(81) Disney, C. M.; Lee, P. D.; Hoyland, J. A.; Sherratt, M. J.; Bay, B. K. A Review of Techniques for Visualising Soft Tissue Microstructure Deformation and Quantifying Strain Ex Vivo. J. Microsc. 2018, 272 (3), 165-179. https://doi.org/10.1111/jmi.12701.
(82) Kurniawan, N. A.; Bouten, C. V. C. Mechanobiology of the Cell-Matrix Interplay: Catching a Glimpse of Complexity via Minimalistic Models. Extreme Mech. Lett. 2018, 20, 59-64. https://doi.org/10.1016/j.em1.2018.01.004.
(83) Caliari, S. R.; Vega, S. L.; Kwon, M.; Soulas, E. M.; Burdick, J. A. Dimensionality and Spreading Influence MSC YAP/TAZ Signaling in Hydrogel Environments. Biomaterials 2016, 103, 314-323. https://doi org/10.1016/j.biomaterials 0.2016.06.061.
(84) Khetan, S.; Guvendiren, M.; Legant, W. R.; Cohen, D. M.; Chen, C. S.; Burdick, J. A. Degradation-Mediated Cellular Traction Directs Stem Cell Fate in Covalently Crosslinked Three-Dimensional Hydrogels. Nat Mater 2013, 12 (5), 458-465.
(85) Lee, H.; Gu, L.; Mooney, D. J.; Levenston, M. E.; Chaudhuri, O. Mechanical Confinement Regulates Cartilage Matrix Formation by Chondrocytes. Nat. Mater. 2017, 16 (12), 1243-1251. https://doi.org/10.1038/nmat4993.
(86) Chaudhuri, O.; Gu, L.; Klumpers, D.; Darnell, M.; Bencherif, S. A.; Weaver, J. C.; Huebsch, N.; Lee, H.; Lippens, E.; Duda, G. N.; Mooney, D. J. Hydrogels with Tunable Stress Relaxation Regulate Stem Cell Fate and Activity. Nat. Mater. 2016, 15 (3), 326-334.
(87) Loebel, C.; Mauck, R. L.; Burdick, J. A. Local Nascent Protein Deposition and Remodelling Guide Mesenchymal Stromal Cell Mechanosensing and Fate in Three-Dimensional Hydrogels. Nat. Mater. 2019, 18 (8), 883-891. https://doi.org/10.1038/s41563-019-0307-6.
(88) Lou, J.; Stowers, R.; Nam, S.; Xia, Y.; Chaudhuri, O. Stress Relaxing Hyaluronic Acid-Collagen Hydrogels Promote Cell Spreading, Fiber Remodeling, and Focal Adhesion Formation in 3D Cell Culture. Biomaterials 2018, 154, 213-222. https://doi.org/10.1016/j.biomaterials.2017.11.004.
(89) Aachmann, F. L.; Larsen, K. L.; Wimmer, R. Interactions of Cyclodextrins with Aromatic Amino Acids: A Basis for Protein Interactions. J. Incl. Phenom. Macrocycl. Chem. 2012, 73 (1), 349-357. https://doi.org/10.1007/s1084′7-011-0071-y.

It will be understood that various details of the presently disclosed subject matter may be changed without departing from the scope of the presently disclosed subject matter. Furthermore, the foregoing description is for the purpose of illustration only, and not for the purpose of limitation.

1. An injectable fibrous hydrogel, the injectable fibrous hydrogel comprising a guest macromer of a hyaluronic acid (HA) backbone and host macromer of a HA backbone, wherein the guest macromer comprises a HA electrospun hydrogel nanofiber functionalized with adamantane (Ad), wherein the host macromer comprises a HA electrospun hydrogel nanofiber functionalized with β-cyclodextrin (CD).

2. The injectable fibrous hydrogel of claim 1, wherein the HA of the guest macromer and the HA of the host macromer comprises a methacrylated HA (MeHA), wherein the MeHA is Ad-modified to form Ad-MeHA in the guest macromer, wherein the MeHA is CD-modified to form CD-MeHA in the host macromer.

3. The injectable fibrous hydrogel of claim 2, wherein the methacrylated HA of the electrospun hydrogel nanofibers is covalently photocrosslinked in the presence of a photoinitiator via ultraviolet (UV) light-mediated radical polymerization.

4. The injectable fibrous hydrogel of claim 1, wherein the guest macromer and host macromer are both hydrophobic and form a stable supramolecular, yet reversible, guest-host interaction.

5. The injectable fibrous hydrogel of claim 1, wherein the hydrogel nanofibers are configured to imbibe water upon hydration rather than dissolving.

6. The injectable fibrous hydrogel of claim 1, wherein the guest macromer and host macromer have a molar ratio ranging from about 1:1 to about 3:1, optionally about 2:1.

7. The injectable fibrous hydrogel of claim 1, wherein the guest macromers and host macromers of the hydrogel nanofibers associate via hydrophobic supramolecular interactions to form a mechanically robust 3D fibrous hydrogel configured for shear-thinning and self-healing post injection.

8. The injectable fibrous hydrogel of claim 7, wherein the guest macromers and host macromers of the injectable fibrous hydrogel have an association constant (Ka) of about 1×104 M−1 to about 1×105 M−1, optionally at least about 1×105 M−1.

9. The injectable fibrous hydrogel of claim 1, wherein the fibrous hydrogel is flowable through a needle at about 8 mL h−1 to about 20 mL h−1 (using a 16 to 22 gauge needle), optionally at about 12 mL h−1 (using a 16 gauge needle), wherein the fibrous hydrogel is configured to transform to a stable hydrogel plug post injection.

10. The injectable fibrous hydrogel of claim 1, wherein the injectable fibrous hydrogel comprises a self-assembling guest-host fibrous hydrogel configured as a cell carrier for injectable tissue engineering.

11. The injectable fibrous hydrogel of claim 1, wherein the injectable fibrous hydrogel is configured to mimic an extra cellular matrix (ECM) upon injection, optionally wherein the injectable fibrous hydrogel is configured to form a hierarchical assembly upon injection to provide physical cues to cells at different length scales, mimicking the 3D cues provided by a native fibrous ECM.

12. The injectable fibrous hydrogel of claim 1, wherein the injectable fibrous hydrogel further comprises one or more ligands, optionally one or more cell adhesion peptides, optionally a cell adhesion peptide comprising arginylglycylaspartic acid (RGD) to permit integrin-mediated cell adhesion.

13. An injectable formulation, the injectable formulation comprising an injectable fibrous hydrogel of claim 1.

14. The injectable formulation of claim 13, further comprising one or more cells encapsulated in the fibrous hydrogel.

15. The injectable formulation of claim 14, wherein the one or more cells have a post-injection survival rate of at least about 70%, optionally at least about 80%, optionally at least about 90%.

16. The injectable formulation of 13, wherein the injectable formulation is configured for injection into a tissue of a subject, optionally a fibrous tissue, optionally a muscle, tendon, or ligament tissue.

17. The injectable formulation of claim 13, wherein the fibrous hydrogel is flowable through a needle at about 12 mL h−1 (16 gauge needle), wherein the fibrous hydrogel is configured to transform to a stable hydrogel plug post injection.

18. The injectable formulation of claim 13, wherein the injectable formulation comprises one or more pharmaceutically acceptable carriers or excipients.

19. The injectable formulation of claim 13, wherein the injectable formulation comprises a storage modulus (G′) of about 6.6 kPa at 1% fibrous content at 10 Hz, and a G′ of about 9.2 kPa at 5% fibrous content at 10 Hz.

20. A method of forming a fibrous hydrogel nanofiber in a polymer solution, the method comprising:
methacrylating a hyaluronic acid (HA) backbone via methacrylate esterification with a primary hydroxyl group of a sodium HA to form methacrylated HA (MeHA);
synthesizing adamantane (Ad)-modified MeHA (Ad-MeHA) and 8-cyclodextrin (CD)-modified MeHA (CD-MeHA) by anhydrous coupling;
electrospinning the fibrous hydrogel nanofiber in the polymer solution; and
crosslinking the fibrous hydrogel nanofiber by exposure to ultraviolet (UV) light.

21. The method of claim 20, wherein a degree of methacrylate modification of HA is controlled by an amount of a methacrylic anhydride introduced during the methacrylating step, optionally wherein the degree of methacrylate modification of HA is about 10% to about 40%, optionally about 20% to about 30%, optionally about 28%.

22. The method of claim 20, wherein Ad-MeHA is prepared using 1-adamantane acetic acid via di-tert-butyl bicarbonate (BOC2O)/4-dimethylaminopyridine (DMAP) esterification, wherein CD-MeHA is prepared using CD-HDA via (benzotriazol-1-yloxy) tris(dimethylamino) phosphonium hexafluorophosphate (BOP) amidation.

23. The method of claim 20, wherein the electrospinning comprises a collection plate set-up using an applied voltage of about 9.5-10.5 kV, a distance from needle to collector of about 16 cm, a needle gauge of about 20, and a flow rate of about 0.4 mL h−1.

24. The method of claim 20, wherein crosslinking the fibrous hydrogel nanofiber with UV light comprises exposure to UV light at about 320-390 nm for about 10-15 minutes.

25. The method of claim 20, wherein the method further comprises repeatedly triturating the hydrogel fibers via needle extrusion to produce short fiber segments of a length of about 5 μm to about 20 μm, optionally about 12.7±5.0 μm.

26. A method of treating a tissue of a subject, the method comprising providing a subject to be treated and delivering to a tissue of the subject an injectable fibrous hydrogel of claim 1.

27. The method of claim 26, wherein the injectable fibrous hydrogel is administered to the tissue to be treated by injection.

28. The method of claim 26, wherein the tissue to be treated is selected from a fibrous tissue, optionally a muscle, tendon, or ligament tissue.

29. The method of claim 28, wherein the injectable fibrous hydrogel comprises one or more encapsulated cells.

30. The method of claim 29, wherein the encapsulated cells have a higher viability post-injection when encapsulated in the fibrous hydrogel than when not encapsulated in the fibrous hydrogel, optionally a survivability rate of at least about 80%.

31. The method of claim 26, wherein the subject to be treated is suffering from a musculoskeletal condition or disease.
